(19) United States
(12) Patent Application Publication
(10) Pub. No.: US 2023/0317285 A1
(43) Pub. Date: Oct. 5, 2023

(54) SYSTEMS AND METHODS FOR DETECTING AND TRACING INDIVIDUALS EXHIBITING SYMPTOMS OF INFECTIONS

(71) Applicant: SIGNIFY HOLDING B.V., EINDHOVEN (NL)

(72) Inventors: DAKSHA YADAV, BOSTON, MA (US); JASLEEN KAUR, MELROSE, MA (US); SHAHIN MAHDIZADEHAGHDAM, MILPITAS, CA (US)

(21) Appl. No.: 18/023,045

(22) PCT Filed: Aug. 9, 2021
(86) PCT No.: PCT/EP2021/072158

§ 371 Date: Feb. 24, 2023

Related U.S. Application Data
(60) Provisional application No. 63/070,518, filed on Aug. 26, 2020.

(30) Foreign Application Priority Data
Sep. 22, 2020 (EP) 20197477.1

(51) Int. Cl. G16H 50/20; G16H 50/80

(52) U.S. Cl. CPC G16H 50/20; G16H 50/80

(57) ABSTRACT

Systems for detecting and localizing a person exhibiting a symptom of infection in a space are provided. The systems include a user interface configured to receive position information of the space and a plurality of connected sensors in the space, wherein the plurality of connected sensors are configured to capture sensor signals from the person exhibiting the symptom of infection. The systems further include a processor configured to input captured sensor signals from the plurality of connected sensors to at least one convolutional neural network (CNN) model selected based on a confidence value, wherein the processor is further configured to locate the symptomatic person. The systems further include a graphical user interface connected to the processor and configured to display the location of the person exhibiting the symptom of infection within the space.

### FIELD OF THE DISCLOSURE

The present disclosure is directed generally to systems and methods for detecting and tracking individuals who exhibit symptoms of illnesses for effective resource management in commercial and/or public settings. More specifically, the present disclosure is directed to systems and methods for detecting individuals exhibiting symptoms of illnesses which can cause the body to exert sounds or movements by integrating audio and video sensors and tracking the individuals using video frames using an internet of things (IoT) system.

### BACKGROUND

Various respiratory ailments are common across the population in different regions of the world. For example, influenza is a contagious respiratory viral disease that typically affects the nose, throat, and lungs of the patient. The novel coronavirus (COVID-19) pandemic also causes symptoms such as cough, shortness of breath, and sore throat and the symptoms may be exhibited 2-14 days after exposure to the virus. Since these diseases are highly contagious and people may be unaware of their infection, it is critical to develop systems and methods for detecting these symptoms quickly and accurately.

In busy environments, such as supermarkets and airports, some people may show symptoms like coughing and sneezing, which can be concerning to others. In this regard, detecting and sanitizing the potentially infected areas can be a store or public place policy. Additionally, mandatory prevention regulations may be implemented by the government. Unfortunately, enforcing such rules in public places such as airports, supermarkets, and train stations can be technically challenging. One challenging aspect is notifying authorities and the public as soon as possible for prevention and social distancing.

Accordingly, there is an urgent need in the art for improved systems and methods for detecting and tracking individuals who exhibit symptoms of illnesses (e.g., viral and bacterial infections and respiratory illnesses) in commercials and/or public settings and notifying authorities and the public.

### SUMMARY OF THE INVENTION

The present disclosure is directed to inventive systems and methods for localizing and tracking sources of coughs, sneezes, and other symptoms of contagious infections for effective resource management in commercial settings. Generally, embodiments of the present disclosure are directed to improved systems and methods for detecting individuals exhibiting symptoms of respiratory illness by integrating audio and video sensors in an internet of things (IoT) system and tracking the individuals using video frames. Applicant has recognized and appreciated that using audio signals without complementary sources of input data can be insufficient to detect symptoms such as sneeze and coughs especially when the audio signals are noisy. Various embodiments and implementations herein are directed to methods of identifying symptoms using audio signals from microphones and, when the audio data is insufficient, using additional signals from cameras and thermopile sensors to identify the symptoms. The microphones, cameras, and thermopile sensors are integrated in or added to light emitting devices in a connected network of multiple devices in an indoor facility. Deep-leaning models are trained for different symptoms to identify potential symptoms and, later uses feature aggregation techniques to reduce the need for labelled samples of the symptoms to be identified. The connected lighting systems can provide visual notifications as soon as symptoms are detected. Authorities can be notified for automatic cleaning or disinfection and/or other appropriate actions.

Generally, in one aspect, a system for detecting and localizing a person exhibiting a symptom of infection in a space is provided. The system includes a user interface configured to receive position information of the space and a plurality of connected sensors in the space. The plurality of connected sensors are configured to capture sensor signals related to the person. The system further includes a processor associated with the plurality of connected sensors and the user interface, wherein the processor is configured to detect whether the person exhibits the symptom of infection based at least in part on captured sensor signals from the plurality of connected sensors and at least one convolutional neural network (CNN) model of first, second, and third CNN models, the at least one CNN model selected based on a confidence value associated with an output of the first CNN model. The processor is further configured to locate the person exhibiting the symptom of infection in the space. The system further includes a graphical user interface connected to the processor and configured to display the location of the person exhibiting the symptom of infection within the space.

In embodiments, the system further includes an illumination device in communication with the processor, wherein the illumination device is arranged in the space and configured to provide at least one light effect to notify others of the location of the person exhibiting the symptom of infection in the space.

In embodiments, the light effect comprises a change in color.

In embodiments, the output of the first CNN model includes a first predicted label and an associated confidence value that at least meets a first predetermined threshold value, and the at least one CNN model includes the first CNN model, wherein the processor is configured to input the captured sensor signals from a first type of sensors of the plurality of connected sensors to the first CNN model.

In embodiments, the output of the first CNN model includes the first predicted label and an associated confidence value that does not at least meet the first predetermined threshold value but at least meets a second predetermined threshold value that is less than the first predetermined threshold value, and the at least one CNN model includes the second CNN model, wherein the processor is configured to input the captured sensor signals from first and second types of sensors of the plurality of connected sensors to the second CNN model. In embodiments, the processor is configured to fuse the captured sensor signals from the first and second types of sensors such that part of the signals from the second type of sensors complements the signals from the first type of sensors.

In embodiments, the output of the first CNN model includes the first predicted label and an associated confidence value that does not at least meet the second predetermined threshold value, and the at least one CNN model includes the third CNN model, wherein the processor is configured to input the captured sensor signals from the second type of sensors of the plurality of connected sensors to the third CNN model.

In embodiments, as partly mentioned, the first type of sensors are different to the second type of sensors. For example, the first type of sensors may be audio sensors, while the second type of sensors may be video sensors, or thermal sensors.

In embodiments, the illumination device may be a luminaire. In embodiments, the illumination device may be configured to maintain the at least one light effect until a disinfection action is determined. For example, the processor according to the invention, or a different processor in communication with the illumination device, may be configured to determine a disinfection action and convey a signal to the illumination device indicative of said disinfection action, wherein the illumination device may receive said signal and stop providing said at least one light effect, or wherein said signal may be configured to control said illumination device to stop providing said at least one lighting effect. Hence, said signal may be a “turn off the at least one light effect” control signal. This enables that the system will not render the at least one light effect, when a disinfection action is determined, and the space is considered safe from a possible infection. Generally, in another aspect, a method for identifying one or more persons exhibiting one or more symptoms of infection in a space is provided. The space includes a plurality of connected sensors configured to capture sensor signals related to the one or more persons. The method includes: requesting infectious symptom presence information from a system having a processor configured to determine whether the one or more persons in the space exhibits one or more symptoms of infection; receiving, by a user interface of a mobile device associated with a user, an input from the user, wherein the input includes a first user tolerance level; and receiving, by the user interface of the mobile device associated with the user, an indication that at least one of the persons within the space exhibits the one or more symptoms of infection. The indication is based on a confidence level selected according to the first user tolerance level. The system is configured to detect whether the one or more persons exhibits the one or more symptoms of infection based at least in part on captured sensor signals from the plurality of connected sensors and at least one convolutional neural network (CNN) model of first, second, and third CNN models, the at least one CNN model selected based on a confidence value associated with an output of the first CNN model.

In embodiments, the method further includes receiving, by the user interface of the mobile device associated with the user, a location of the one or more persons detected as exhibiting the one or more symptoms of infection in the space; and providing at least one light effect by an illumination device in communication with the processor of the system to notify others of the location of the one or more persons exhibiting the one or more symptoms of infection in the space.

In embodiments, the output of the first CNN model includes a first predicted label and an associated confidence value that at least meets a first predetermined threshold value, and the at least one CNN model includes the first CNN model, wherein the at least one processor is configured to input the captured sensor signals from a first type of sensors of the plurality of connected sensors to the first CNN model.

In embodiments, the output of the first CNN model includes the first predicted label and an associated confidence value that does not at least meet the first predetermined threshold value but at least meets a second predetermined threshold value that is less than the first predetermined threshold value, and the at least one CNN model includes the second CNN model, wherein the at least one processor is configured to input the captured sensor signals from first and second types of sensors of the plurality of connected sensors to the second CNN model.

In embodiments, the output of the first CNN model includes the first predicted label and an associated confidence value that does not at least meet the second predetermined threshold value, and the at least one CNN model includes the third CNN model, wherein the at least one processor is configured to input the captured sensor signals from the second type of sensors of the plurality of connected sensors to the third CNN model.

In embodiments, the method further includes the step of changing, by the user interface, the first user tolerance level to a second user tolerance level that is different than the first user tolerance level.

In embodiments, the method further includes the steps of receiving, by the user interface of the mobile device associated with the user, a location of the one or more persons detected as exhibiting the one or more symptoms of infection in the space; and rendering, via the user interface, at least one route within the space that avoids the location of the one or more persons detected as exhibiting the one or more symptoms of infection in the space.

Generally, in yet a further aspect, a method of determining whether a person exhibits symptoms of an infection is provided. The method includes receiving samples from a positive class of a new symptom, samples from a negative class of the new symptom, and a query signal; extracting, by a feature extraction module, features from the samples of the positive class of the new symptom, the samples from the negative class of the new symptom, and the query signal; aggregating, by a feature aggregation module, the features from the samples of the positive class of the new symptom with the query signal to generate a positive class feature representation; aggregating, by the feature aggregation module, the features from the samples of the negative class of the new symptom with the query signal to generate a negative class feature representation; receiving, by a comparison module, the positive class feature representation and the negative class feature representation; and determining, by the comparison module, whether the query signal is more similar to the positive class feature representation or the negative class feature representation.

In various implementations, the processor described herein may take any suitable form, such as, one or more processors or microcontrollers, circuitry, one or more controllers, a field programmable gate array (FGPA), or an application-specific integrated circuit (ASIC) configured to execute software instructions. Memory associated with the processor may take any suitable form or forms, including a volatile memory, such as random-access memory (RAM), static random-access memory (SRAM), or dynamic random-access memory (DRAM), or non-volatile memory such as read only memory (ROM), flash memory, a hard disk drive (HDD), a solid-state drive (SSD), or other non-transitory machine-readable storage media. The term “non-transitory” means excluding transitory signals but does not further limit the forms of possible storage. In some implementations, the storage media may be encoded with one or more programs that, when executed on one or more processors and/or controllers, perform at least some of the functions discussed herein. It will be apparent that, in embodiments where the processor implements one or more of the functions described herein in hardware, the software described as corresponding to such functionality in other embodiments may be omitted. Various storage media may be fixed within a processor or may be transportable, such that the one or more programs stored thereon can be loaded into the processor so as to implement various aspects as discussed herein. Data and software, such as the algorithms or software necessary to analyze the data collected by the tags and sensors, an operating system, firmware, or other application, may be installed in the memory.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the present disclosure.

FIG. 1 is an example flowchart showing systems and methods for localizing and tracking a symptomatic person in a space according to aspects of the present disclosure;

FIG. 1A is an example schematic depiction of a lighting IoT system for localizing and tracking a symptomatic person in a space according to aspects of the present disclosure;

FIG. 2 is an example flowchart showing adaptive selection of a CNN model based on a confidence value of the dynamic symptom detection system of FIG. 1 according to aspects of the present disclosure;

FIG. 3 is an example flowchart showing how the CNN models of FIG. 2 are used to determine whether a person exhibits symptoms of an infection with fewer samples according to aspects of the present disclosure;

FIG. 4 is an example process for determining whether a person exhibits symptoms of an infection with a CNN model using fewer samples according to aspects of the present disclosure;

FIG. 4A is an example process for determining whether a person exhibits symptoms of an infection using fewer samples according to aspects of the present disclosure;

FIG. 5 is an example flowchart showing using video frames for tracking a symptomatic person with CNNs and RNNs according to aspects of the present disclosure;

FIG. 6 is a schematic depiction of a connected lighting system using light effects to indicate which areas are safe and which should be avoided or approached with caution according to aspects of the present disclosure;

FIG. 7 is an example of a user interface device that can be used for visualization of a symptomatic person in the space according to aspects of the present disclosure;

FIG. 8 is an example user interface for setup and configuration of proposed systems according to aspects of the present disclosure;

FIG. 9 is an example user interface for setup and configuration of proposed systems according to aspects of the present disclosure;

FIG. 10 is an example user interface configured to display locations where symptomatic people were detected according to aspects of the present disclosure;

FIG. 11 is an example user interface configured to display a location where a potentially infected person is situated and a corresponding confidence level associated with the prediction according to aspects of the present disclosure; and

FIG. 12 is an example process for detecting and localizing one or more persons exhibiting one or more symptoms of infection in a space according to aspects of the present disclosure.

### DETAILED DESCRIPTION OF EMBODIMENTS

The present disclosure describes various embodiments of systems and methods for detecting and tracking symptomatic individuals in commercial settings by integrating audio and video sensors in connected lighting systems and tracking the symptomatic individuals using video frames. Applicant has recognized and appreciated that it would be beneficial to identify symptoms using a dynamic symptom detection system which utilizes an appropriate convolutional neural network (CNN) which is selected based on confidence value. The different CNNs are trained on different data and, in such a way that they require fewer training samples. Thus, the CNNs can be quickly adapted for new symptoms. Applicant has also recognized and appreciated that it would be beneficial to utilize a trained CNN in conjunction with recurrent neural networks to track the symptomatic individuals. Notifications can be sent to property managers or administrators to take appropriate action in embodiments of the present disclosure. Appropriate actions may include targeted disinfection, restricting access to a particular area of concern, etc. Notifications can also be provided to others in the vicinity of the symptomatic individuals using light effects provided by the connected lighting systems.

The present disclosure describes various embodiments of systems and methods for providing a distributed network of symptom detection and tracking sensors by making use of illumination devices that are already arranged in a multi-grid and connected architecture (e.g., a connected lighting infrastructure). Such existing infrastructures can be used as a backbone for the additional detection, tracking, and notification functionalities described herein. Signify's SlimBlend® suspended luminaire is one example of a suitable illumination device equipped with integrated IoT sensors such as microphones, cameras, and thermopile infrared sensors as described herein. In embodiments, the illumination device includes USB type connector slots for the receivers and sensors etc. Illumination devices including sensor ready interfaces are particularly well suited and already provide powering, digital addressable lighting interface (DALI) connectivity to the luminaire's functionality and a standardized slot geometry. It should be appreciated that any illumination devices that are connected or connectable and sensor enabled including ceiling recessed or surface mounted luminaires, suspended luminaires, wall mounted luminaires, and free floor standing luminaires, etc. are contemplated. Suspended luminaires or free floor standing luminaires including thermopile infrared sensors are advantageous because the sensors are arranged closer to humans and can detect higher temperatures of people. Additionally, the resolution of the thermopile sensor can be lower than for thermopile sensors mounted within a ceiling recessed or surface mounted luminaire mounted at approximately 3 m ceiling height.

The term “luminaire” as used herein refers to an apparatus including one or more light sources of same or different types. A given luminaire may have any one of a variety of mounting arrangements for the light source(s), enclosure/housing arrangements and shapes, and/or electrical and mechanical connection configurations. Additionally, a given luminaire optionally may be associated with (e.g., include, be coupled to and/or packaged together with) various other components (e.g., control circuitry) relating to the operation of the light source(s). Also, it should be understood that light sources may be configured for a variety of applications, including, but not limited to, indication, display, and/or illumination.

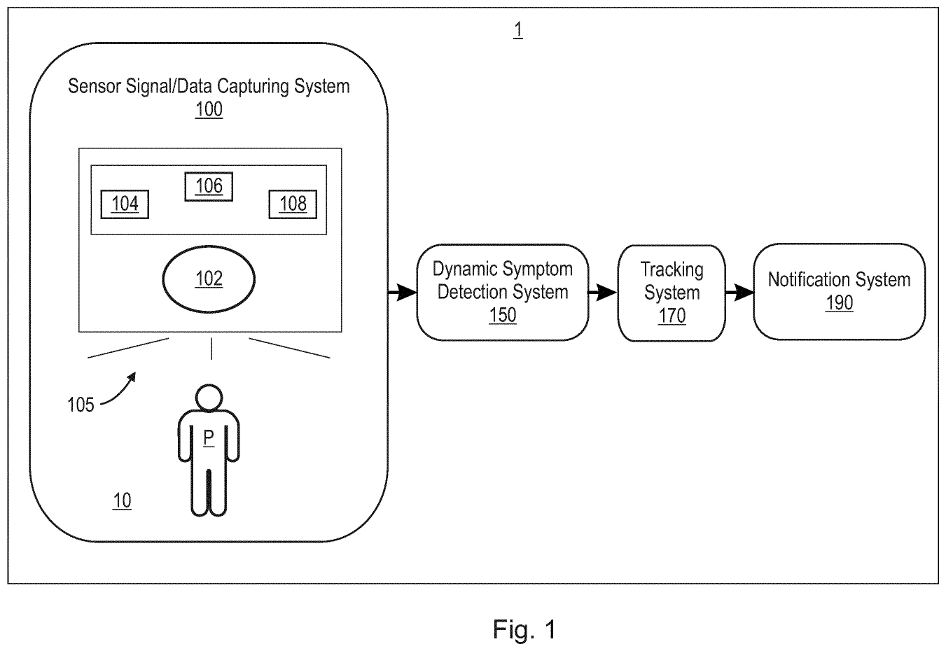

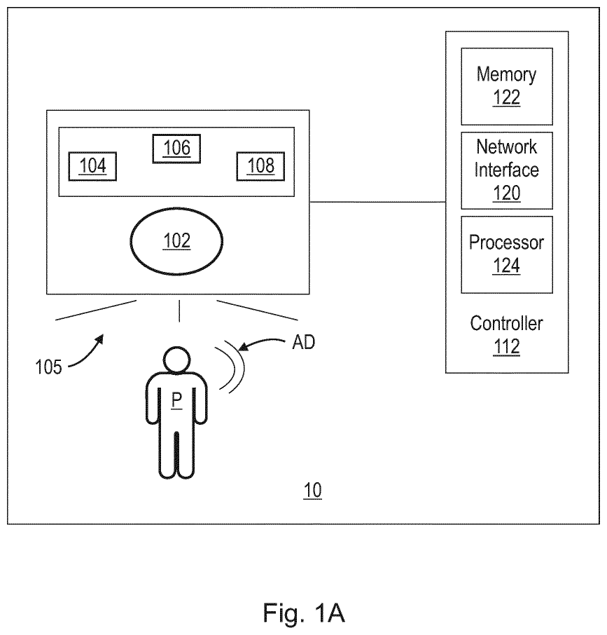

Referring to FIG. 1, a schematic depiction of a flowchart showing systems and methods for localizing and tracking a symptomatic person in a space is provided. The flowchart includes a system 1 for detecting and localizing a person P exhibiting a symptom of infection in a space 10, the system 1 including sensor signal and data capturing system 100, a dynamic symptom detection system 150, a tracking system 170, and a notification system 190. The sensor signal and data capturing system 100 includes a connected lighting system including illumination devices 102 and on-board sensors such as microphone sensors 104, image sensors 106 (e.g., cameras), and multiple-pixel thermopile infrared sensors 108. It should be appreciated that the on-board sensors can also include ZigBee transceivers, Bluetooth® radio, light sensors, and IR receivers in embodiments. The dynamic symptom detection system 150 is configured to dynamically select the source of input (audio, audio plus complementary video data, or video data) from the system 100 and input the selected signals to the appropriate convolutional neural network (CNN) model. In embodiments, there are three separate CNN models: one for audio input, one for audio plus complementary video input, and another for video input and, each is trained for symptom detection such that they require fewer training samples than traditional CNN models. The tracking system 170 is configured to detect and localize symptomatic individuals using video frames. The notification system 190 is configured to use the connected lighting system infrastructure to notify the building managers as well as other occupants in the vicinity. These systems and methods are described in more detail below.

The sensor signal and data capturing system 100 is embodied as a lighting IoT system for symptom localization in a space 10. The system 100 includes one or more overhead connected lighting networks that are equipped with connected sensors (e.g., advanced sensor bundles (ASBs)). The overhead connected lighting networks refer to any interconnection of two or more devices (including controllers or processors) that facilitates the transmission of information (e.g., for device control, data storage, data exchange, etc.) between the two or more devices coupled to the network. Any suitable network for interconnecting two or more devices is contemplated including any suitable topology and any suitable communication protocols. The sensing capabilities of the ASBs are used to accurately detect and track symptomatic individuals within a building space 10. It should be appreciated that the lighting IoT system 100 can be configured in a typical office setting, a hotel, a grocery store, an airport, or any suitable alternative.

The lighting IoT system 100 includes illumination devices 102 that may include one or more light-emitting diodes (LEDs). The LEDs are configured to be driven to emit light of a particular character (i.e., color intensity and color temperature) by one or more light source drivers. The LEDs may be active (i.e., turned on); inactive (i.e., turned off); or dimmed by a factor d, where 0≤d≤1. The value d=0 means that the LED is turned off whereas d=1 represents an LED that is at its maximum illumination. The illumination devices 102 may be arranged in a symmetric grid or, e.g., in a linear, rectangular, triangular or circular pattern. Alternatively, the illumination devices 102 may be arranged in any irregular geometry. It should be appreciated that the overhead connected lighting networks include the illumination devices 102, microphone sensors 104, image sensors 106, thermopile sensors 108 among other sensors of the ASBs to provide a sufficiently dense sensor network to cover a whole building indoor space. Although in some embodiments the illumination devices 102, microphone sensors 104, image sensors 106, and thermopile sensors 108 are all integrated together and configured to communicate within a single device via wired or wireless connections, in other embodiments any one or more of the microphone sensors 104, image sensors 106, and thermopile sensors 108 can be separate from the illumination devices 102 and in communication with the illumination devices 102 via a wired or wireless connection.

The illumination devices 102 are arranged to provide one or more visible lighting effects 105 which can include a flashing of the one or more LEDs and/or one or more changes of color of the one or more LEDs. A flashing of the one or more LEDs can include activating the one or more LEDs at a certain level at regular intervals for a period of time and deactivating or dimming the one or more LEDs a certain amount between the regular intervals when the LEDs are active. It should be appreciated that, when flashing, the LEDs can be active at any specific level or a plurality of levels. It should also be appreciated that the LEDs can flash at irregular intervals and/or increasing or decreasing lengths of time. The one or more LEDs can also or alternatively provide a visible lighting effect including one or more changes of color. The color changes can occur at one or more intensity levels. The illumination devices 102 can be controlled by a central controller 112 as shown in FIG. 1A. For example, as described herein the controller 112 can control the illumination devices 102 together or individually based on where person P is determined to be located after the system determines person P to have exhibited symptoms of a respiratory illness. In example embodiments, the controller 112 can cause the LEDs of the illumination devices 102 to change from a default setting to one or more colors indicative of a level of caution needed to be exercised in that area. For example, if the system determines that person P is symptomatic with a 50% confidence level, the illumination devices surrounding person P can be configured to change to a yellow color. If the system determines that person P is symptomatic with a 95% confidence level, the illumination devices surrounding person P can be configured to change to a red color. It should be appreciated that any colors can be used instead of yellow and red as described. Additionally, the spectral power distribution of the LEDs can be adjusted by the controller 112. Any suitable lighting characteristic can be controlled by controller 112.

Controller 112 includes a network interface 120, a memory 122, and one or more processors 124. Network interface 120 can be embodied as a wireless transceiver or any other device that enables the connected luminaires to communicate wirelessly with each other as well as other devices including mobile device 700 utilizing the same wireless protocol standard and/or to otherwise monitor network activity and enables the controller 112 to receive data from the connected sensors 104, 106, and 108. In embodiments, the network interface 120 may use wired communication links. The memory 122 and one or more processors 124 may take any suitable form in the art for controlling, monitoring, and/or otherwise assisting in the operation of illumination devices 102 and performing other functions of controller 112 as described herein. The processor 124 is also capable of executing instructions stored in memory 122 or otherwise processing data to, for example, perform one or more steps of the methods described herein. Processor 124 may include one or more modules, such as, a data capturing module of system 100, a dynamic symptom detection module of system 150, a tracking module of system 170, a notification module of system 190, and the feature extraction 208, feature aggregation 210 and comparison 212 modules of system 200.

As shown in FIGS. 1 and 1A, the microphone sensors 104, the camera sensors 106, and the multi-pixel thermopile sensors 108 are configured to detect sensor signals from person P exhibiting signs of an illness. Microphone sensors 104 can capture audio data AD from sounds from person P. Camera sensors 106 can capture video data of person P. Thermopile sensors 108 can capture temperature-sensitive radiation from person P. Additional sensors can be used as well. For example, one or more forward-looking infrared (FLIR) thermal cameras can be used to measure the body temperature of person P. Since the illumination devices and microphone, camera, and thermopile sensors are arranged at specific fixed locations within the space, position information of their fixed locations can be stored locally and/or at memory 122.

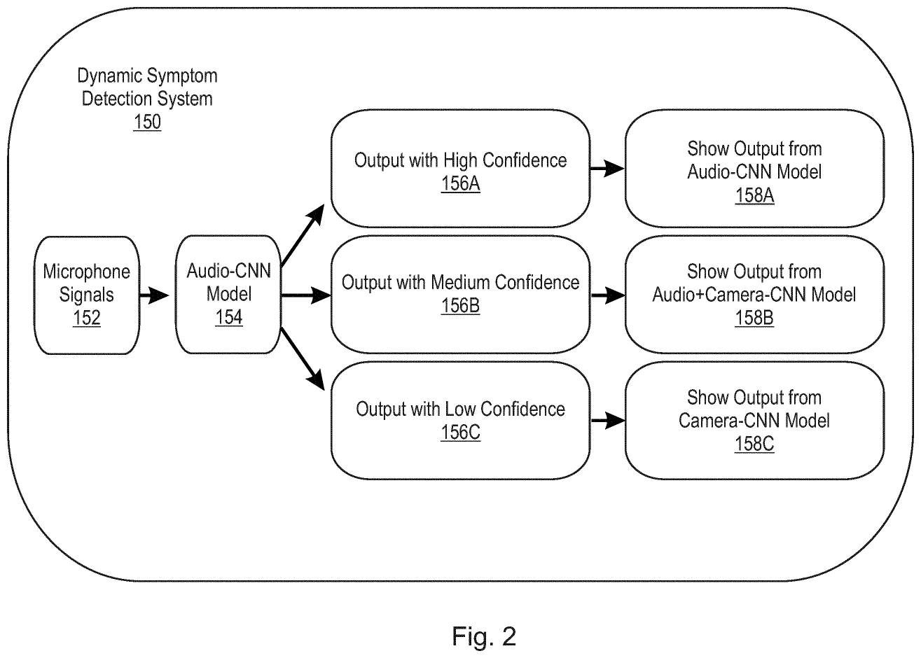

As shown in FIG. 2, the dynamic symptom detection system 150 relies on data captured from the lighting IoT system 100 to perform a binary classification in determining whether the captured data indicates a symptom or not. Specifically, the dynamic symptom detection system 150 uses the microphone signals as an input to an audio-CNN model 154 for the binary classification. The audio-CNN model 154 outputs a predicted label (e.g., a symptom or not) along with a confidence value which indicates the model's confidence in the predictable label. Using this confidence value, there are the following three scenarios:

The first scenario occurs when the audio-CNN model 154 outputs a predicted label with a high confidence value 156A. When the model is highly confident about its prediction, the system uses this output as is (e.g., the system outputs the results of the binary classification of the audio-CNN model 158A). In embodiments, the high confidence value 156A can be measured against a predetermined threshold value. If the confidence value 156A is equal to or above the predetermined threshold value, then the confidence value 156A qualifies as a high confidence value or a sufficiently confident value. A sufficiently confident value means that the audio signals are sufficient by themselves to form a symptom prediction.

The second scenario occurs when the audio-CNN model 154 outputs a predicted label with a medium confidence value 156B. In other words, in the second scenario, the audio-CNN model 154 outputs a predicted label with a confidence value that is less than the high confidence value in the first scenario. For example, the confidence value 156B can be less than the predetermined threshold value discussed in the first scenario and equal to or above another lower predetermined threshold value indicative of a low confidence level. If the confidence value 156B is below the predetermined threshold value used in the first scenario and above another predetermined threshold value used to indicate a low confidence level, then the confidence value 156B qualifies as a medium confidence value. In this scenario, the audio signals AD are fused together with data from the cameras and this fused data is sent to an audio+camera-CNN model for the binary classification. In this second scenario, the system outputs the results of the binary classification of the audio+camera-CNN model 158B. It should be appreciated that in embodiments, the amount of camera data used is limited to an amount necessary to complement the audio data rather than the full camera data. This second scenario can be particularly advantageous when the audio signal may be noisy, and the model confidence can be improved by leveraging additional data from the camera.

The third scenario occurs when the audio-CNN model 154 outputs a predicted label with a low confidence value 156C. In other words, in the third scenario, the audio-CNN model 154 outputs a predicted label with a confidence value that is less than the lower predetermined threshold value that indicates a low confidence level discussed in the second scenario. If the confidence level 156C is below the lower predetermined threshold value, then the confidence value 156C qualifies as a low confidence value and the audio data is insufficient to make any conclusions about the symptom. In this scenario, the data from the cameras is used instead of the audio data. The camera data is sent to a camera-CNN model for the binary classification. In this third scenario, the system outputs the results of the binary classification of the camera-CNN model 158C.

As shown above, using the dynamic symptom detection system 150 provides an agile, adaptive, and precise localization of a potentially symptomatic person.

The audio-CNN model, the audio+camera-CNN model, and the camera-CNN model have an improved architecture when compared with typical CNN architectures such as the Oxford Visual Geometry Group (VGG), Inception, etc. Typical CNN architectures require a large amount of training data to achieve their accuracy levels. However, such large amounts of training data may not be available to train symptom classification and may require a significant amount of time to train. In the present disclosure, the audio-CNN model, the audio+camera-CNN model, and the camera-CNN model are trained with only a few samples of a positive class (exhibiting at least one symptom).

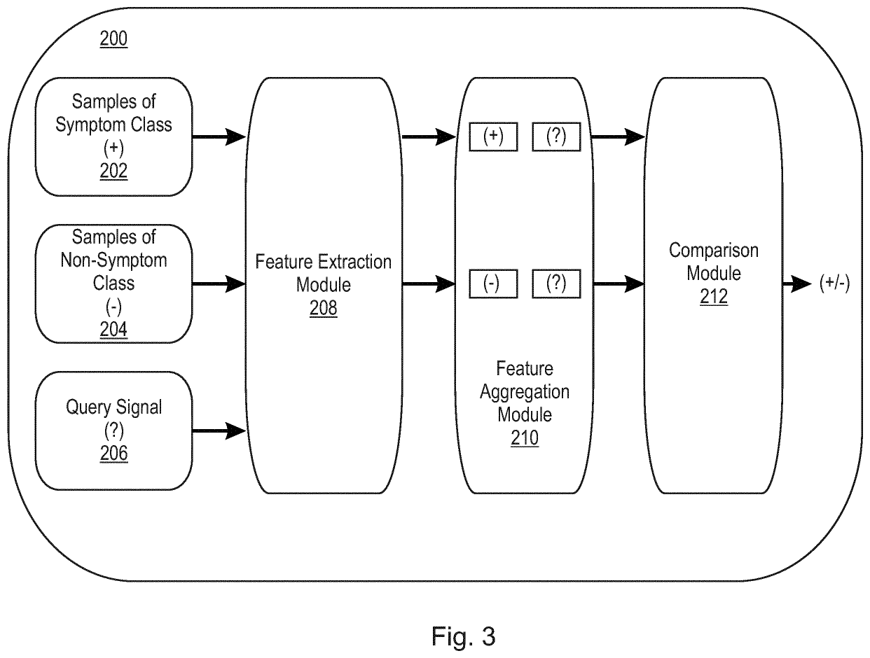

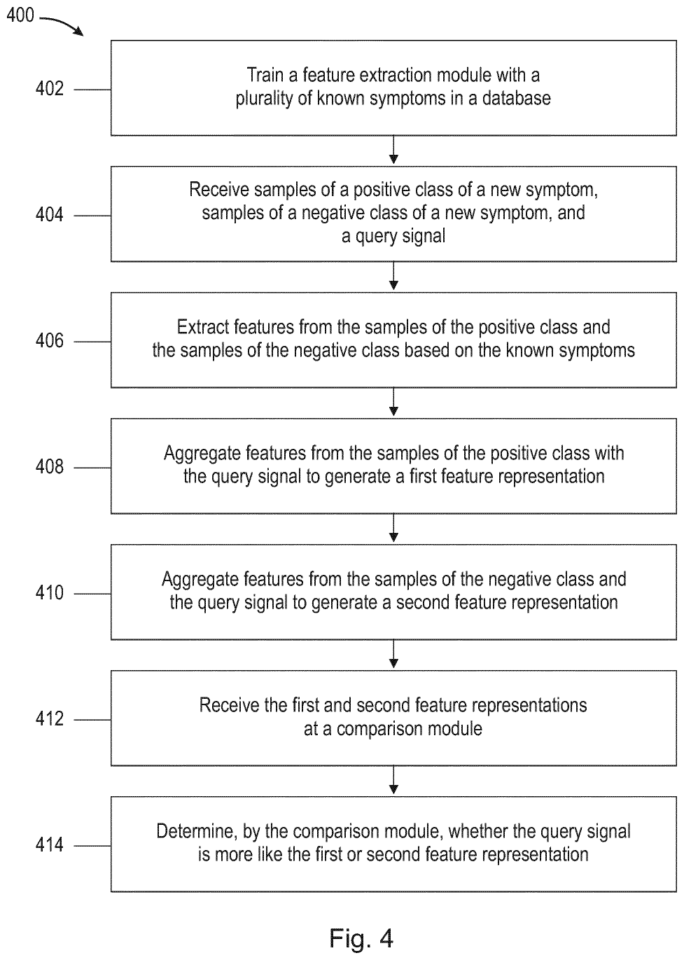

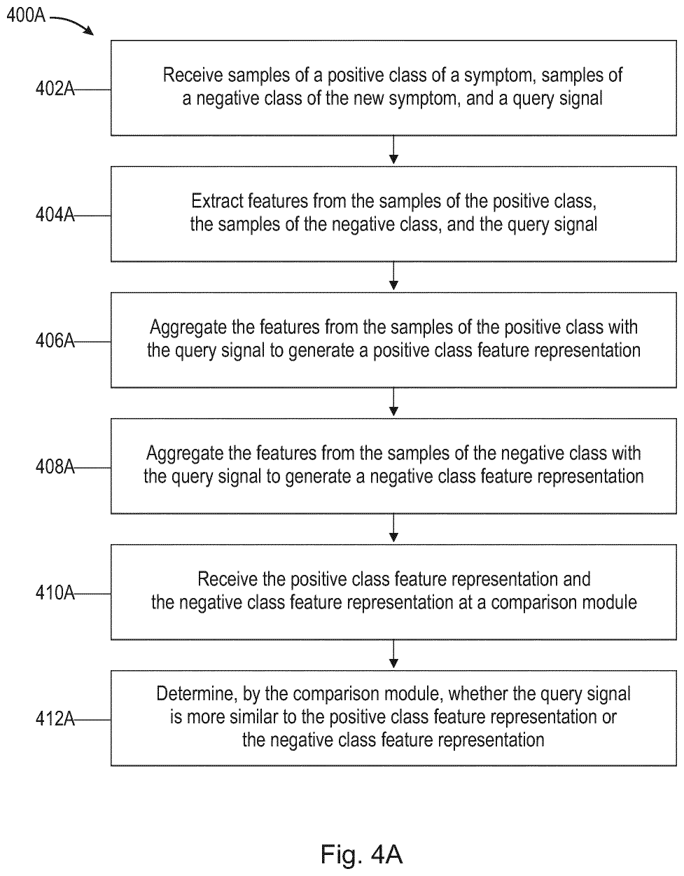

The following should be appreciated in view of FIGS. 3 and 4 which show processes 200 and 400 of using the CNN models of FIG. 2 to determine whether a person exhibits symptoms of an infection with fewer samples. In the first step, samples from a positive class (+) 202, samples from a negative class (−) 204, and a query signal (?) 206 (e.g., an audio signal of a potential symptom) are sent to a feature extraction module 208. It should be appreciated that the query signal (?) is an audio signal of a potential symptom for the audio-CNN model but that the query signal (?) for the audio+camera-CNN model is a signal of fused audio and camera data of a potential symptom and the query signal (?) for the camera-CNN model is a camera signal of a potentially symptomatic person. The samples from the positive class (+) 202 include features indicative of actual symptoms whereas the samples from the negative class (−) 204 do not have such features. Thus, it should be appreciated that, for the audio-CNN model, the samples from the positive class (+) 202 are samples including audio signals having features of at least one actual symptom, for the audio+camera-CNN model, the samples from the positive class (+) 202 are samples including fused audio and camera data having features of at least one actual symptom, and for the camera-CNN model, the samples from the positive class (+) 202 are samples including camera data having features of at least one actual symptom. The samples from the negative class (−) 204 for the audio-CNN model, the audio+camera-CNN model, and the camera-CNN model do not include the features of actual symptoms found in the samples of the positive classes.

As shown in FIG. 4, the feature extraction module 208 can be trained using a plurality of known symptoms in a database at step 402. After the feature extraction module is trained, at step 404, the feature extraction module 208 is configured to receive the samples from a positive class (+) 202, samples from a negative class (−) 204, and the query signal (?) 206 as discussed above. At step 406, the feature extraction module 208 is configured to extract features from the samples of the positive and negative classes based on the known symptoms. At steps 408 and 410, the feature aggregation module 210 creates two feature representations: one feature representation of aggregated features from the samples of the positive class and the query signal and another feature representation of aggregated features from the samples of the negative class and the query signal. In other words, features from the samples of the positive class are aggregated with the query signal to generate a first feature representation and features from the samples of the negative class are aggregated with the query signal to generate a second feature representation.

These two sets of features are then sent to a comparison module 212 comprising various convolutional layers. At step 412, a comparison module 212 is configured to receive the first and second feature representations and, at step 414, the comparison module 212 is configured to determine whether the query signal is more like or similar to the first feature representation or the second feature representation. Due to this formulation of combining positive and negative features with the query, training the CNN models requires significantly fewer samples to learn whether the query is closer to the positive class (symptom) or the negative class (others without symptoms).

As shown in FIG. 4A, an example process 400A of determining whether a person exhibits symptoms of an infection is provided. The method starts with receiving 402A samples from a positive class of a new symptom 202, samples from a negative class of the new symptom 204, and a query signal 206. At step 404A, the method involves extracting, by a feature extraction module 208, features from the samples of the positive class, the samples of the negative class, and the query signal. At step 406A, the method involves aggregating, by a feature aggregation module 210, the features from the samples of the positive class with the query signal to generate a positive class feature representation. The method further involves aggregating, by the feature aggregation module 210, the features from the samples of the negative class with the query signal to generate a negative class feature representation at step 408A. At step 410A, the method includes receiving, by a comparison module 212, the positive class feature representation and the negative class feature representation. Lastly, at step 412A, the method includes determining, by the comparison module 212, whether the query signal is more similar to the positive class feature representation or the negative class feature representation.

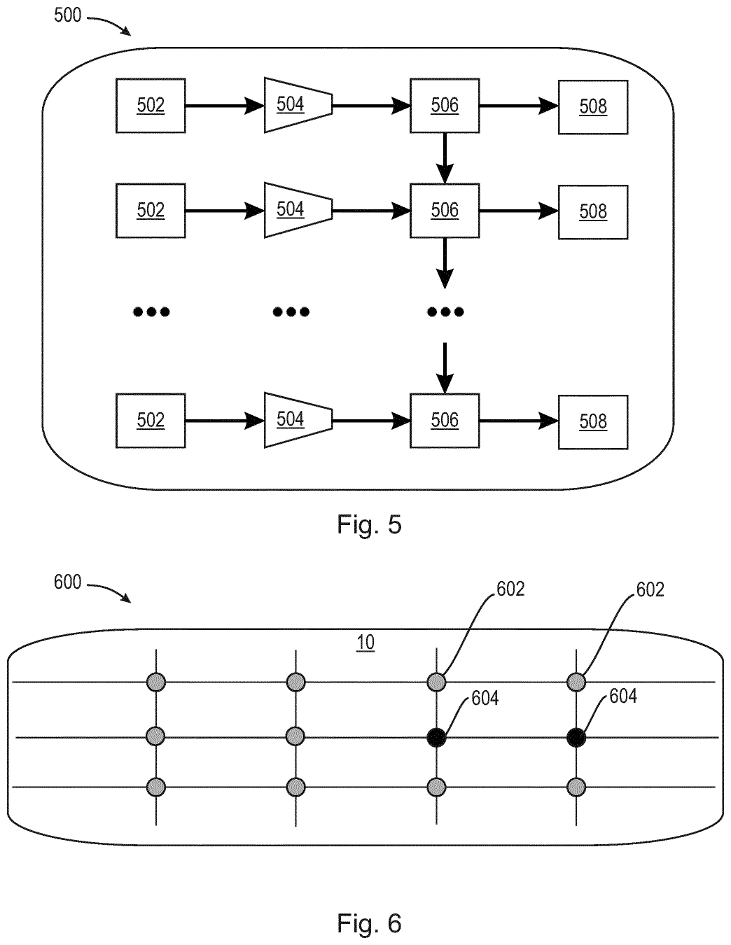

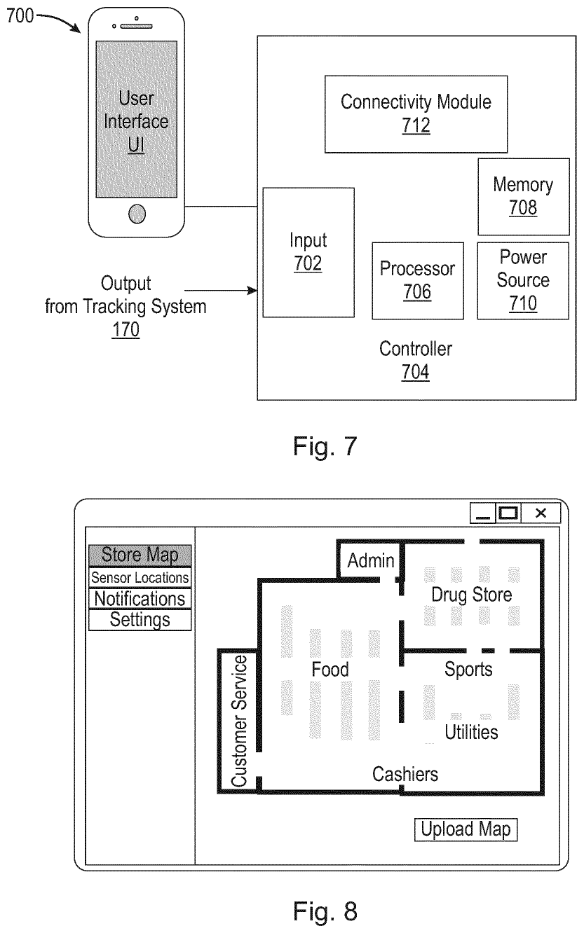

When the dynamic symptom detection system 150 reveals that a symptom is predicted in a space, the camera data can be used for monitoring the source of that symptom as shown in the architecture 500 in FIG. 5. Architecture 500 is part of tracking system 170 described above. In embodiments, deep learning models that are trained for people tracking are used to perform feature extraction on video frames 502. The feature extraction models 504 can be initialized from pre-trained activity detection models (such as VDETLIB as described in “Object Detection From Video Tubelets with Convolutional Neural Networks”, Kang et al. In Proceedings of the IEEE Conference on Computer Vision and Pattern Recognition (pp. 817-825)) and fine-tuned with limited training samples. Later, the extracted features from each video frame are fed into a Recurrent Neural Network 506 to localize the position of the symptomatic person. In Recurrent Neural Networks (RNNs), connections between nodes are in a temporal sequence, which allows it to exhibit temporal dynamic behaviors. Since the RNN modules 506 are linked together, the proposed architecture 500 can track a symptomatic individual for a few consecutive sequences of frames and identify the actions as a cough, sneeze, etc.

As shown in FIG. 6, the lighting IoT system can have embedded sensors as discussed above and can be configured to signal to other occupants in the space which areas within the space are safe to use and navigate and which areas within the space should be avoided or approached with caution. The illumination devices of FIG. 6 are part of the notification system 190 described above. As shown in FIG. 6, each connected illumination device can be associated with one or more particular areas within the space 10. One or more particular illumination devices 602 can be controlled by controller 112 to emit a specific color or series of colors to indicate whether the corresponding area within the space is safe to use and navigate. In other words, depending on the location of the symptomatic person determined with the tracking system 170, the illumination devices 602 of the notification system 190 can illuminate selected areas within the space 10 in a particular color (e.g., default white, green, or yellow) to indicate those areas are safe or symptom free. The illumination devices 604 of the notification system 190 can also illuminate selected areas within the space 10 in a particular color (e.g., red or orange) to indicate those areas are not safe or not symptom free. The illumination devices 602 and 604 can be configured to illuminate the selected areas within the space 10 at a regular interval in embodiments. The illumination devices 602 and 604 can also be configured to illuminate the selected areas within the space 10 when a symptomatic person is predicted with the dynamic symptom detection system 150 and localized with the tracking system 170. In other embodiments, the illumination devices 602 and 604 can be configured to illuminate the selected areas within the space on demand (e.g., from a user about to enter or already within the space). In example embodiments, based on the colors emitted by illumination devices 602 and 604, authorities and/or facility managers can be prompted to take action, e.g., perform disinfection routines or removal of a symptomatic person. Because the change in the color of the light denotes an area where certain unwanted activity such as a cough or sneeze is detected, such remedial action can be taken quickly and accurately. Other occupants can also take extra precautions when entering the space with red lights overhead.

As described herein, the sensors of the lighting IoT system 100 are configured to transmit audio data, fused audio/camera data, and/or camera data to processor 124 via any suitable wired/wireless network communication channels. In embodiments, the sensor data can be transmitted directly to computer processor 124 without passing through a network. The sensor data can be stored in memory 122 via the wired/wireless communication channels. Particular embodiments of the present disclosure are useful as an administrator user interface for an administrator in charge of the space. Other particular embodiments of the present disclosure are useful for other occupants within the space.

In the embodiments for an administrator and/or other occupants, system 100 can additionally include any suitable device 700 as part of the notification system 190. The suitable device 700 is capable of receiving user input and executing and displaying a computer program product in the form of a software application or a platform. Device 700 can be any suitable device, such as, a mobile handheld device, e.g., a mobile phone, a personal computer, a laptop, a tablet, or any suitable alternative. The software application can include a user interface (UI) configured to receive and/or display information useful to the administrator as described herein. In an example, the software application is an online application that enables an administrator to visualize the location of a symptomatic person detected with the dynamic symptom detection system 150 and localized with tracking system 170 in the space 10. The device 700 includes an input 702, a controller 704 with a processor 706 and a memory 708 which can store an operating system as well as sensor data and/or output data from the CNN models, and/or output from the tracking system 170. The processor 706 is configured to receive output from the tracking system 170 described herein via the input 702. The output from tracking system 170 can be stored in memory 708. In embodiments, device 700 can also be used to transmit sensor data within the sensor signal/data capturing system 100 via any Internet of Things system. The device 700 can also include a power source 710 which can be AC power, or can be battery power from a rechargeable battery. The device can also include a connectivity module 712 configured and/or programmed to communicate with and/or transmit data to a wireless transceiver of controller 122. In embodiments, the connectivity module can communicate via a Wi-Fi connection over the Internet or an Intranet with memory 122, processor 124, or some other location. Alternatively, the connectivity module may communicate via a Bluetooth or other wireless connection to a local device (e.g., a separate computing device), memory 122, or another transceiver. For example, the connectivity module can transmit data to a separate database to be stored or to share data with other users. In embodiments, the administrator can verify the location of the symptomatic person and use the device 700 to cause the controller 122 to control the illumination devices 102 as described herein (e.g., to change colors in particular areas). In embodiments, the administrator can cause the controller 122 to control the illumination devices 102 to display default settings (e.g., a default color) after the appropriate cleaning protocols have been completed.

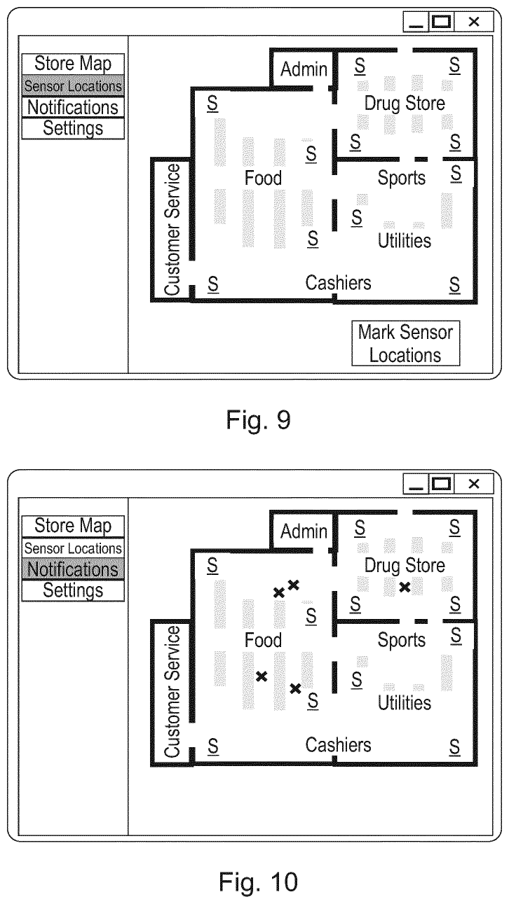

In embodiments for an administrator, device 700 includes UI associated with the processor 706. Floor plan information of the space 10 can be provided by an administrator via UI as shown in FIG. 8. The floor plan information can be embodied as an image uploaded to device 700. In alternate embodiments, the floor plan information can be retrieved from memory 708 via a system bus or any suitable alternative. UI may include one or more devices or software for enabling communication with an administrator-user. The devices can include a touch screen, a keypad, touch sensitive and/or physical buttons, switches, a keyboard, knobs, levers, a display, speakers, a microphone, one or more indicator lights, audible alarms, a printer, and/or other suitable interface devices. The user interface can be any device or system that allows information to be conveyed and/or received, and may include a graphical display configured to present to an administrator user views and/or fields configured to receive entry and/or selection of information. For example, as shown in FIGS. 8 and 9 an administrator user can use UI for an initial configuration and installation of the framework. As shown, the UI can provide functionalities for setting floor plan information of the space 10, sensor locations, and default parameters. The initial configuration is performed in the space 10 in embodiments. The UI may be located within one or more components of the system (e.g., processor 706) or may be located remote from the system 100 and in communication with the system via wired/wireless communication channels. In FIG. 9, the administrator can input position information of the sensors S within the floor plan of the space 10 via UI. In alternate embodiments, the position information of the sensors S can be retrieved from memory 708 or memory 122. As shown in FIG. 10, notifications of notification system 190 can be displayed to an administrator via UI. In embodiments, each “X” depicted in FIG. 10 indicates a symptomatic person detected with the dynamic symptom detection system 150 and localized with tracking system 170. Using the UI shown in FIG. 10, an administrator can visualize the locations of any potential infection transfer and implement necessary disinfection protocols at these areas.

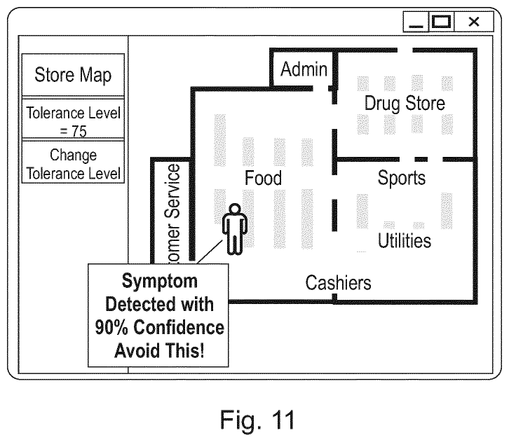

In embodiments for other occupants of the space 10, UI of device 700 can be configured such that the other occupants of the space can interact with the systems described herein. As shown in FIG. 11, when a customer/user enters space 10 with the floor plan information and sensor information as described above, they can utilize UI of device 700 to visualize other occupants as well as symptom detection predictions. Further, they can also visualize the confidence value associated with each symptom detection prediction. As shown in FIG. 11, another occupant is visible in the space 10 along with a notation that a symptom is detected to have been emanated from the occupant using the dynamic symptom detection system described above. The notation also includes a confidence value (e.g., 90%) associated with the symptom detection prediction from the dynamic symptom detection system described above.

In embodiments, the occupant interacting with the UI of FIG. 11 can input their tolerance level and/or change their tolerance level. The tolerance level can be directly related to their perceived level of health or immunity. As shown in FIG. 11, the user has input a tolerance level of 75 out of a range from 0-100. It should be appreciated that the tolerance level can be a numeric value as shown in FIG. 11 or it could be a percentage value or some range of values. In embodiments, the tolerance level could also be a non-numeric scale such as an ordinal scale indicating the user's tolerance or comfort level. If the user inputs a tolerance level of 0 out of a range from 0-100, then the user means they have no tolerance for any amount of potential infection transfer. If the user inputs a tolerance level of 0, then the UI will display all occupants deemed to be a source of a predicted symptom regardless of the confidence level. If the user inputs a tolerance level of 100 out of a range from 0-100, then the user means they can tolerate any amount of potential infection transfer. If the user inputs a tolerance level of 100, then the UI will not display any occupants deemed to be a source of a predicted symptom regardless of the confidence level.

In embodiments, the UI of FIG. 11 is configured to display occupants deemed to be the source of a predicted symptom when the confidence value associated with the symptom prediction is equal to or above the user's tolerance level. The tolerance levels can be associated with the confidence values in a one-to-one relationship. Thus, in example embodiments, a tolerance level of 50 corresponds to a 50% confidence level, a tolerance level of 65 corresponds to a 65% confidence level, and so on. In example embodiments, a tolerance level of 5 out of a range from 1 to 10 corresponds to 50-59% confidence levels within a range of 0-100%. Thus, a single tolerance level can correspond to multiple confidence levels. In example embodiments, a tolerance range can be provided (e.g., 50-75) and such a range can correspond to 50-75% confidence levels within a range of 0-100% or 30-45 where the confidence value range is smaller, e.g., 0-60. Thus, the tolerance value ranges can be equal to the confidence value ranges or the tolerance value ranges can be smaller or larger than the confidence value ranges.

In FIG. 11, an occupant in the space is displayed to the user with a notation that the occupant is the source of a predicted symptom since the confidence value associated with the predicted symptom is 90% and 90% is above the user's tolerance level of 75. If two occupants are displayed via the UI in the space and both occupants are deemed to be the sources of predicted symptoms, both can be displayed with the same or different confidence values associated with the symptom predictions so long as the values are equal to or higher than the user's tolerance level. For example, one notation can have a confidence value that is higher than the confidence value associated with the other notation. If one of the two confidence values is 75% and the other is 95%, the user can decide that the area with the confidence value of 75% is less risky than the area with the confidence value of 95%. The user can also decide to avoid the area with the confidence value of 95%. In embodiments, the UI can also be configured to display optimized routes to the user avoiding the areas vulnerable to potential infection transfer.

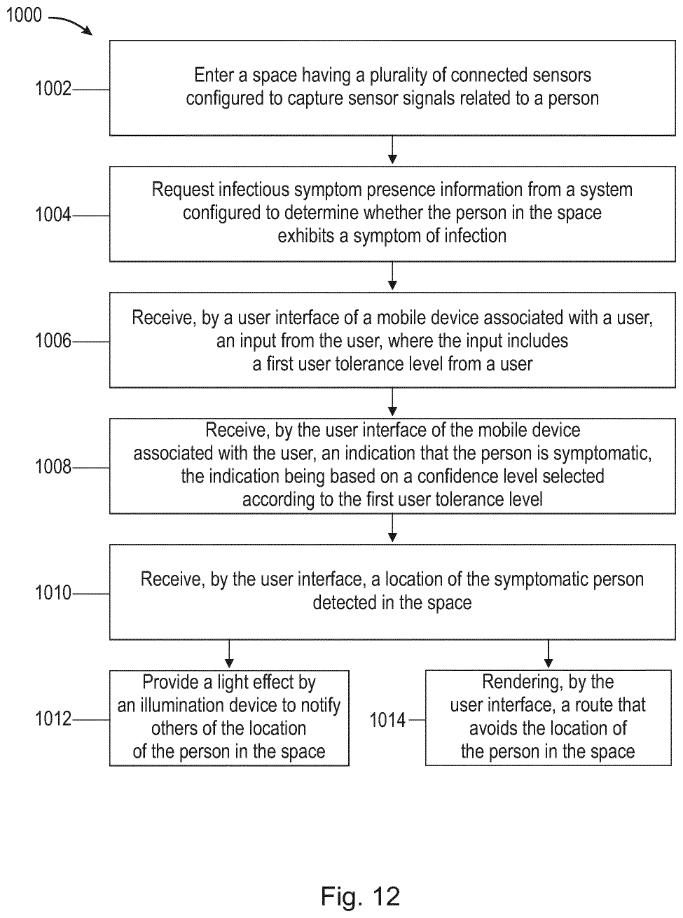

Referring to FIG. 12, a method 1000 for identifying one or more persons exhibiting one or more symptoms of infection in a space is provided. The method begins at step 1002 when a customer/user enters a space having a plurality of connected sensors configured to capture sensor signals related to other occupants in the space. The customer/user enters the space with a mobile device configured to interact with the system 1 described herein.

At step 1004 of the method, the customer/user requests infectious symptom presence information from a system (e.g., system 1) having a processor configured to determine whether the other occupants in the space exhibit symptoms of infection. The system is configured to detect whether other occupants in the space exhibit symptoms based at least in part on captured sensor signals from the connected sensors and at least one convolutional neural network (CNN) model as described above. At least one CNN model of first, second, and third CNN models is selected based on a confidence value associated with an output of the first CNN model.

At step 1006 of the method, the customer/user inputs a first user tolerance level using a UI associated with the mobile device he/she is carrying.

At step 1008 of the method, the customer/user receives, by the UI of the user's mobile device, an indication that at least one of the occupants in the space exhibits a symptom of infection. The indication is based on an associated confidence level from the at least one CNN model and selected according to the first user tolerance level.

At step 1010 of the method, the customer/user receives, by the UI of the user's mobile device, a location of the one or more persons detected as exhibiting the one or more symptoms of infection in the space.

At step 1012 of the method, at least one light effect is provided by an illumination device in communication with a processor of the system 1 to notify others of the location of the one or more persons detected as exhibiting the one or more symptoms of infection in the space.

At step 1014 of the method, the customer/user receives, by the UI of the user's mobile device, at least one route within the space that avoids the location of the one or more persons exhibiting the one or more symptoms of infection in the space.

Advantageously, the systems and methods described herein provide for improved localizing and tracking of a symptomatic person by utilizing connected sensors such as microphones and cameras and a dynamic symptom detection system. The dynamic symptom detection system utilizes a convolutional neural network (CNN) model which is selected by confidence value. The different CNNs are trained on microphone signals, camera data, or a fusion of microphone and camera signals. The CNNs are trained in such a fashion that they require fewer training samples and hence, can be quickly adapted for new symptoms which do not have sufficiently large training data. Once an instance of a symptom is detected, the symptomatic person can be tracked using a CNN model trained for tracking people in conjunction with recurrent neural networks. Notifications can be sent to property managers or administrators to take appropriate action. Notifications can also be sent to people sharing the space with the symptomatic individual.

It should also be understood that, unless clearly indicated to the contrary, in any methods claimed herein that include more than one step or act, the order of the steps or acts of the method is not necessarily limited to the order in which the steps or acts of the method are recited.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A system for detecting and localizing a person exhibiting a symptom of infection in a space, comprising:
a user interface configured to receive position information of the space and a plurality of connected sensors in the space, wherein the plurality of connected sensors are configured to capture sensor signals related to the person;
a processor associated with the plurality of connected sensors and the user interface, wherein the processor is configured to detect whether the person exhibits the symptom of infection based at least in part on captured sensor signals from the plurality of connected sensors and at least one convolutional neural network (CNN) model of first, second, and third CNN models, the at least one CNN model selected based on a confidence value associated with an output of the first CNN model, wherein the processor is further configured to locate the person exhibiting the symptom of infection in the space; and
a graphical user interface connected to the processor and configured to display the location of the person exhibiting the symptom of infection within the space;
wherein the processor is configured to input the captured sensor signals from a first type of sensors of the plurality of connected sensors to the first CNN model;
wherein the processor is configured to input the captured sensor signals from first and second types of sensors of the plurality of connected sensors to the second CNN model;
wherein the processor is configured to input the captured sensor signals from the second type of sensors of the plurality of connected sensors to the third CNN model.

2. The system of claim 1, further comprising:
an illumination device in communication with the processor, wherein the illumination device is arranged in the space and configured to provide at least one light effect to notify others of the location of the person detected as exhibiting the symptom of infection in the space.

3. The system of claim 2, wherein the light effect comprises a change in color.

4. The system of claim 2, wherein the illumination device is a luminaire.

5. The system of claim 1, wherein the output of the first CNN model comprises a first predicted label and an associated confidence value that at least meets a first predetermined threshold value, and the at least one CNN model comprises the first CNN model, wherein the processor is configured to input the captured sensor signals from a first type of sensors of the plurality of connected sensors to the first CNN model.

6. The system of claim 5, wherein the output of the first CNN model comprises the first predicted label and an associated confidence value that does not at least meet the first predetermined threshold value but at least meets a second predetermined threshold value that is less than the first predetermined threshold value, and the at least one CNN model comprises the second CNN model, wherein the processor is configured to input the captured sensor signals from first and second types of sensors of the plurality of connected sensors to the second CNN model.

7. The system of claim 6, wherein the processor is configured to fuse the captured sensor signals from the first and second types of sensors such that part of the signals from the second type of sensors complements the signals from the first type of sensors.

8. The system of claim 6, wherein the output of the first CNN model comprises the first predicted label and an associated confidence value that does not at least meet the second predetermined threshold value, and the at least one CNN model comprises the third CNN model, wherein the processor is configured to input the captured sensor signals from the second type of sensors of the plurality of connected sensors to the third CNN model.

9. A method for identifying one or more persons exhibiting one or more symptoms of infection in a space having a plurality of connected sensors, wherein the plurality of connected sensors are configured to capture sensor signals related to the one or more persons, the method comprising the steps of:
requesting, by a user interface of a mobile device associated with a user, infectious symptom presence information from a system comprising a processor configured to determine whether the one or more persons in the space exhibits one or more symptoms of infection;
receiving, by the user interface of a mobile device associated with a user, an input from the user, wherein the input comprises a first user tolerance level; and
a processor of the system detecting whether the one or more persons exhibits the one or more symptoms of infection based at least in part on captured sensor signals from the plurality of connected sensors and at least one convolutional neural network (CNN) model of first, second, and third CNN models, the at least one CNN model selected based on a confidence value associated with an output of the first CNN model;
wherein the confidence level is selected according to the first user tolerance level;
wherein the processor is configured to input the captured sensor signals from a first type of sensors of the plurality of connected sensors to the first CNN model; wherein the processor is configured to input the captured sensor signals from first and second types of sensors of the plurality of connected sensors to the second CNN model; wherein the processor is configured to input the captured sensor signals from the second type of sensors of the plurality of connected sensors to the third CNN model;
receiving, by the UI of the mobile device associated with the user, from the system, an indication that at least one person of the one or more persons within the space exhibits the one or more symptoms of infection, the indication being based on the confidence level selected according to the first user tolerance level.

10. The method of claim 9, further comprising the steps of:
receiving, by the UI of the mobile device associated with the user, a location of the one or more persons detected as exhibiting the one or more symptoms of infection in the space; and
providing at least one light effect by an illumination device in communication with the processor of the system to notify others of the location of the one or more persons detected as exhibiting the one or more symptoms of infection in the space.

11. The method of claim 9, wherein the output of the first CNN model comprises a first predicted label and an associated confidence value that at least meets a first predetermined threshold value, and the at least one CNN model comprises the first CNN model, wherein the at least one processor is configured to input the captured sensor signals from the first type of sensors of the plurality of connected sensors to the first CNN model.

12. The method of claim 10, wherein the output of the first CNN model comprises the first predicted label and an associated confidence value that does not at least meet the first predetermined threshold value but at least meets a second predetermined threshold value that is less than the first predetermined threshold value, and the at least one CNN model comprises the second CNN model, wherein the at least one processor is configured to input the captured sensor signals from first and second types of sensors of the plurality of connected sensors to the second CNN model.

13. The method of claim 12, wherein the output of the first CNN model comprises the first predicted label and an associated confidence value that does not at least meet the second predetermined threshold value, and the at least one CNN model comprises the third CNN model, wherein the at least one processor is configured to input the captured sensor signals from the second type of sensors of the plurality of connected sensors to the third CNN model.

14. The method of claim 9, further comprising the step of changing, by the user interface, the first user tolerance level to a second user tolerance level that is different than the first user tolerance level.

15. The method of claim 9, further comprising the steps of:
receiving, by the UI of the mobile device associated with the user, a location of the one or more persons detected as exhibiting the one or more symptoms of infection in the space; and
rendering, via the UI of the mobile device associated with the user, at least one route within the space that avoids the location of the one or more persons detected as exhibiting the one or more symptoms of infection in the space.
